(19) United States
(12) Patent Application Publication
(10) Pub. No.: US 2013/0252235 A1
(43) Pub. Date: Sep. 26, 2013

(54) Mobility Controlled Single Macromolecule in Nanofluidic System and its Application as Macromolecule Sequencer

(71) Applicant: Jinyao Tang, Emeryville, CA (US)

(72) Inventor: Jinyao Tang, Emeryville, CA (US)

(21) Appl. No.: 13/615,087

(22) Filed: Sep. 13, 2012

Related U.S. Application Data
(60) Provisional application No. 61/534,874, filed on Sep. 14, 2011.

(51) Int. Cl. C12Q 1/68

(52) U.S. Cl. CPC C12Q 1/6869
USPC 435/6.1; 435/287.2

(57) ABSTRACT

In one embodiment, the present application discloses a microfluidic device comprising at least one nanochannel, in part, configured to receive a macromolecule, at least one external electrodes configured with the nanochannel, that is further configured to provide contact of the macromolecule with an enzyme to cleave the macromolecule to form a fragment of the macromolecule. Also disclosed are methods of using the microfluidic device for detecting and identifying a property of the fragment of the macromolecule.

### RELATED APPLICATIONS

This application claims the benefit of U.S. Provisional Patent Application No. 61/534,874 filed on Sep. 14, 2011, entitled “Mobility Controlled Single Macromolecule in Nanofluidic System and Its Application as Macromolecule Sequencer” which is incorporated herein by reference.

### BACKGROUND OF THE INVENTION

The present application discloses methods for fine control of movement of molecules, including macromolecules, such as an individual macromolecule or a mixture of the macromolecules and fragment or fragments thereof, the design and fabrication of micro or nanometer scale devices and apparatus to realize the control, as well as the application of this method and of these devices and apparatus in sequencing and analyzing properties of polymers and macromolecules.

Rapid and accurate DNA sequencing technology is one of the fundamental driving force for the development of basic biological research, as well as has numerous applications in biotech, drug development, pharmacogenetics and pharmacogenomics, forensic biology and many others technologies. With the advances of modern DNA sequencing technology, complete DNA sequences of many animal, plant and microbial genomes may be obtained. Particularly, DNA sequencing technology includes apparatuses, methods and technologies that are used to determine the order of the nucleotide bases—adenine, guanine, cytosine and thymine—in a molecule of DNA. The history of DNA sequencing technology starts with the first DNA sequence obtained from two-dimensional chromatography in the early 1970s; the dye-based sequencing methods then allowed automated analysis.

DNA sequencing technologies have been made easier to perform, cheaper to obtain and orders of magnitude faster due to the development of high-throughput sequencing technologies that parallelize the sequencing process and produce thousands or millions of sequences at once. At the present time, the dominant technologies available, the “next generation” DNA sequencing technologies, employ different sequencing platforms, such as sequencing-by-synthesis and sequencing-by-ligation, and utilize more advanced technology than the traditional capillary electrophoresis method. However, these technologies have limits in read length, data accuracy, rate of data output and also higher cost due to the need of excessive reagents. For example, the sequencers using microarray technology can only handle limited length of DNA due to yield of chemical reaction; and the data output rate is slow due to limits of chemical reaction rate; and the sequencing is expensive because fluorescent chemicals are needed in most cases.

Third generation DNA sequencing technologies, most of which based on single molecule sequencing, are currently under rapid development to provide quicker and more accurate results with much lower cost. Many proposed and under development sequencers utilize nano structures such as nanochannel, nanopore and nanogap. For example, nanopore-based analysis methods often involve passing a polymeric molecule, for example single-stranded DNA (“ssDNA”), through a nanoscopic opening while monitoring a signal such as an electrical signal. Typically, the nanopore is designed to have a size that allows the polymer to pass only in a sequential, single file order. As the polymer molecule passes through the nanopore, differences in the chemical and physical properties of the monomeric units that make up the polymer, for example, the nucleotides that compose the ssDNA, are translated into characteristic detectable signals, such as electrical and optical signals.

The electrical signal can, for example, be detected as a modulation of the ionic current by the passage of a DNA molecule through the nanopore, which current is created by an applied voltage across the nano structure, such as a nanopore-bearing membrane or film. The optical signal can, for example, be collected by charge-coupled device (CCD). Because of structural differences between different nucleotides, each different type of nucleotide within the ssDNA produces a type-specific modulation as it passes through a nano structure, and thus allowing the sequence of the DNA to be determined.

Devices using nanopores to sequence DNA and RNA molecules have generally not been capable of reading sequence at a single-nucleotide resolution.

The foregoing examples of the related art and limitations are intended to be illustrative and not exclusive. Other limitations of the related art will become apparent to those of skill in the art upon a reading of the specification and a study of the drawings or figures as provided herein.

### SUMMARY OF THE INVENTION

While all the above described developments have shown some capability for detecting some sequence information, we have determined that there is a need for accurate, reliable devices and methods for measuring sequences such as those of RNA and DNA. Similarly, there is also a related need for devices capable of sequencing molecules at a high speed and at a high level of resolution. The following embodiments, aspects and variations thereof are exemplary and illustrative are not intended to be limiting in scope.

In one aspect, there is provided a microfluidic device comprising: at least one nanochannel comprising an inlet channel and an outlet channel, wherein the nanochannel comprises at least one slow zone and a first fast zone configured in the nanochannel; the nanochannel further configured to receive a macromolecule at the inlet channel and the inlet channel configured to be in fluid communication with the slow zone, the first fast zone and the outlet channel; wherein the macromolecule comprises a leading segment, a middle segment and an end segment; at least one external electrode configured with the nanochannel for applying a first gate potential to the slow zone and a second gate potential to the first fast zone of the nanochannel for independently controlling and changing the rate of motion of at least among the leading segment, the middle segment and the end segment of the macromolecule, for controlling the rate of translocation of the macromolecule through the nanochannel and for controlling the rate of motion of a fragment obtained from a cleavage from the macromolecule; the nanochannel further configured to provide contact of the macromolecule with an enzyme to cleave the macromolecule to form a fragment of the macromolecule; and a detector for detecting and identifying a property of the fragment, wherein the detector is configured to detect the fragment in a second fast zone, wherein the fragment detected is obtained in the same sequence as the sequence of the uncleaved macromolecule.

As used herein, a segment is defined as a portion of the macromolecule that may include the leading segment, the middle segment and the end segment of the macromolecule. A fragment of the molecule is defined as a portion of the molecule that has been cleaved or hydrolyzed from macromolecule. A fragment may comprise of a single monomer, such as a single nucleotide or a single base. In another aspect, a fragment may comprise a dimer, trimer, oligomer, residues of protein, other charged fragments of the macromolecules, or mixtures thereof, of the macromolecule. In one embodiment, the enzyme is an exonuclease. In another embodiment, the enzyme is an endonuclease. In another embodiment, the microfluidic device comprises at least two external electrodes.

As used herein, a nanochannel, a nanotube, a microfluidic channel are used interchangeable and may differ depending on the size and configuration of the channel or the tube, as disclosed herein. In one embodiment, the microfluidic device may be an integrated microfluidic system comprising the microfluidic device comprising a plurality nanochannel, macromolecule extractor, such as a nucleic acid extractor, a macromolecule purification system, such as a nucleic acid purification column, an amplification system, such as a nucleic acid amplification system, a sequencing system, such as a nucleic acid sequencer, a detection system such as one or more optical detectors in communication with a control system for collecting and analyzing information from the detectors. In one aspect, the control system provides the sequence of the macromolecule obtained from the microfluidic device.

In one aspect of the above microfluidic device, the application of a first gate field to the slow zone upon the motion of the macromolecule through the nanochannel slows the motion of at least one segment of the macromolecule, and the application of a second gate field to the fast zone increases the motion or speed of at least one segment of the macromolecule, stretching the macromolecule segments between the slow zone and the first fast zone. In another aspect of the above microfluidic device, the fragment of the macromolecule is a monomer. In another aspect of the above device, the macromolecule is DNA.

In one aspect, the macromolecule is selected from the group consisting of DNA, RNA, oligonucleotides, polynucleotides, proteins, carbohydrates or charged polymers, and mixtures thereof. In another aspect, the macromolecule is a single DNA or a single RNA. In another aspect, the macromolecule is a mixture of macromolecules, such as a mixture of different DNA or mixtures of different RNA, and combinations thereof. In another aspect, the macromolecule is charged. In one variation, the macromolecule is negatively charged.

In another aspect of the above microfluidic device, the first gate potential and the second gate potential are different. In another aspect, the detector detects an optical signal capable of detecting a single molecule fluorescence and/or capable of detecting a single molecule Raman signal. In another aspect of the above device, the detector detects the change in the electrical properties. In another aspect, the microfluidic device is configured to provide a controlled single molecule delivery. In yet another aspect of the above device, the property of the fragment is selected from the groups consisting of mechanical strength, length or number of bases/residues in polymers or oligomers and/or in a mixture, transport properties and molecular electronic properties of the macromolecule.

In another embodiment, there is provided a method for obtaining an identity of a target macromolecule comprising: a) introducing the target macromolecule to a microfluidic device, wherein the microfluidic device comprises: at least one nanochannel comprising an inlet channel and an outlet channel, wherein the nanochannel comprises at least one slow zone and a first fast zone configured in the nanochannel; the nanochannel further configured to receive the target macromolecule at the inlet channel and the inlet channel configured to be in fluid communication with the slow zone, the first fast zone and the outlet channel; wherein the target macromolecule comprises a leading segment, a middle segment and an end segment; at least one external electrodes configured with the nanochannel for applying a first gate potential to the slow zone and a second gate potential to the first fast zone of the nanochannel for independently controlling and changing the rate of motion of at least among the leading segment, the middle segment and the end segment of the target macromolecule, for controlling the rate of translocation of the target macromolecule through the nanochannel and for controlling the rate of motion of a fragment obtained from a cleavage from the target macromolecule; the nanochannel further configured to provide contact of the target macromolecule with an exonuclease to cleave the target macromolecule to form a fragment of the target macromolecule; and a detector for detecting and identifying a property of the fragment, wherein the detector is configured to detect the fragment in a second fast zone, wherein the fragment detected is obtained in the same sequence as the sequence of the uncleaved target macromolecule; b) conveying the target macromolecule through the nanochannel by applying an electrical field or a differential pressure; c) applying a first gate potential to the slow zone, and a second, different gate potential to the fast zone to control the relative motion of the leading segment, the middle segment and the end segment of the target macromolecule to stretch or increase the spacing among the segments; d) contacting the stretched segment of the target macromolecule with an enzyme to cleave the target macromolecule to form a cleaved fragment; e) conveying the cleaved fragment to a second fast zone; and 0 detecting the cleaved fragment and determining the sequence of the target macromolecule. In one aspect, the enzyme is an exonuclease or an endonuclease.

In one aspect of the above method, the application of a first gate field to the slow zone upon the motion of the macromolecule through the nanochannel slows the motion of at least one segment of the macromolecule, and the application of a second gate field to the first fast zone increases the speed or motion of at least one segment of the macromolecule, stretching the macromolecule segments between the slow zone and the first fast zone. In another aspect of the method, the increase in speed or motion is about 1000 to 5000 times faster than the speed of the macromolecule in the slow zone. In another aspect, the increase in speed or motion is about 2000 to 3000 times faster than the speed of the macromolecule in the slow zone. In another aspect of the above method, the stretched segment of the macromolecule is increased from about 0.3 nm to about 300 nm-1.5 μm. In another aspect, the stretched segment of the macromolecule is increased from about 100 nm to about 200 nm. In yet another aspect of the above method, the target macromolecule has a translocation speed of about 20 mm/sec and decreases to about 10-20 μm/sec in the slow zone, or about 2-10 μm/sec in the slow zone. In another aspect of the above method, the fragment of the macromolecule is a monomer. In another aspect of the method, the macromolecule is DNA. In yet another aspect of the above method, the first gate potential and the second gate potential are different. In another aspect of the method, the detector detect an optical signal capable of detecting a single molecule fluorescence and/or capable of detecting a single molecule Raman signal. In another aspect of the method, the detector is a fluorescence detector, a CCD detector or a combination of both detectors.

In one aspect of the above method, the detector is an ionic current detector and detects the ionic current or changes in the ionic current. In another aspect, the nanochannel is decorated, coated or impregnated with functional groups on the surface of the nanochannel, and the detection and characterization of the fragments, such as monomers, oligomers, individual bases or individual nucleotide, may be performed by passing the fragments through the above functionalized nanochannels. For example, a series of individual nucleotides, obtained in the original order or sequence in the DNA molecule, may pass through the nanochannel using the above cited methods. The strength of interactions between the decorated groups and different types of fragments, for example nucleotide bases—adenine, guanine, cytosine and thymine, will be type-specific, for example the modulation of the motion or the speed of different nucleotide will be different, which can allow the fragments to be identified. For example, the modification may be one type of nucleotide base-adenine, guanine, cytosine and thymine or the combination of the nucleotides which will specifically interact with different complementary or non-complementary nucleotide.

In another aspect of the method, the detector detects the change in the electrical properties. In yet another aspect, the microfluidic device is configured to provide a controlled single molecule delivery. In another aspect of the method, the property of the fragment is selected from the groups consisting of mechanical strength, length or number of bases/residues in polymers or oligomers and/or in a mixture, transport properties, molecular electronic properties of the macromolecule. In another aspect of the method, the nanochannel is positively charged. In another aspect, the exonuclease cleaves the macromolecule at a rate of >1 kbp/s, >2 kbp/s, >5 kbp/s or 10 kbp/s.

In addition to the exemplary embodiments, aspects and variations described above, further embodiments, aspects and variations will become apparent by reference to the drawings and figures and by examination of the following descriptions.

### DETAILED DESCRIPTION OF THE INVENTION

### Description of the Figures

FIG. 1. A schematic representation of the mobility of DNA molecule inside a nanochannel modulated with external gates and sequencing devices. As shown in FIG. 1, A; a negatively charged DNA molecule passes through a positively charged nanotube. Mobility can be modulated by surface charge on the sidewall of nanotube and/or by external potential as a driving force. As shown in FIG. 1, B; a DNA may be stretched when passing through the first two zones, a slow zone and a fast zone. If labeled by fluorophore, DNA motion inside the external gated nanochannel can be observed in-situ. The stretched DNA molecule is then conveyed to the exonuclease with slow rate. The well separated individual bases can be resolved readily with single molecule sensitive detection technique such as fluorescent microscopy.

FIG. 2. Shows schematic representative factors that may affect the macromolecule mobility in the nanochannel. Higher surface charge density may lead to stronger molecule to surface interaction and lower mobility, while other factors may also affect the DNA mobility. Such factors may include the electrophoresis field, the solution concentration, the surface coatings, etc. and combinations thereof.

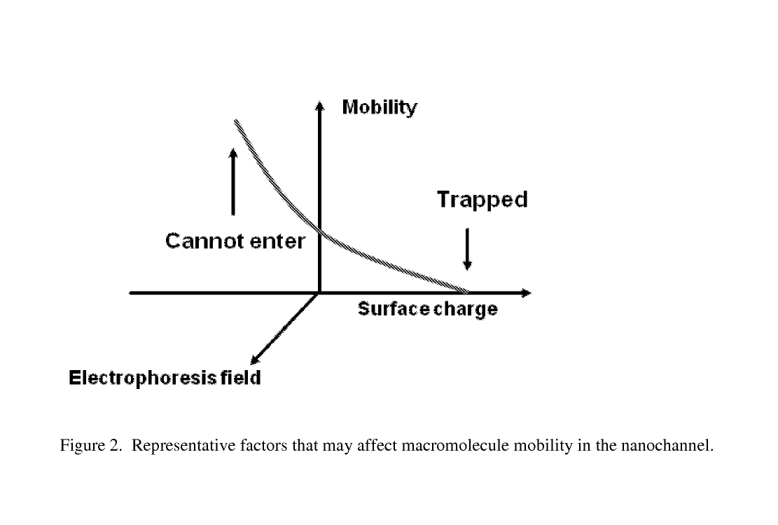

FIG. 3. Discloses a representative schematic of a single molecule DNA sequencer, including a nanochannel having a slow zone (with low speed), a fast zone (with high speed), and a detector such as a fluorescence camera.

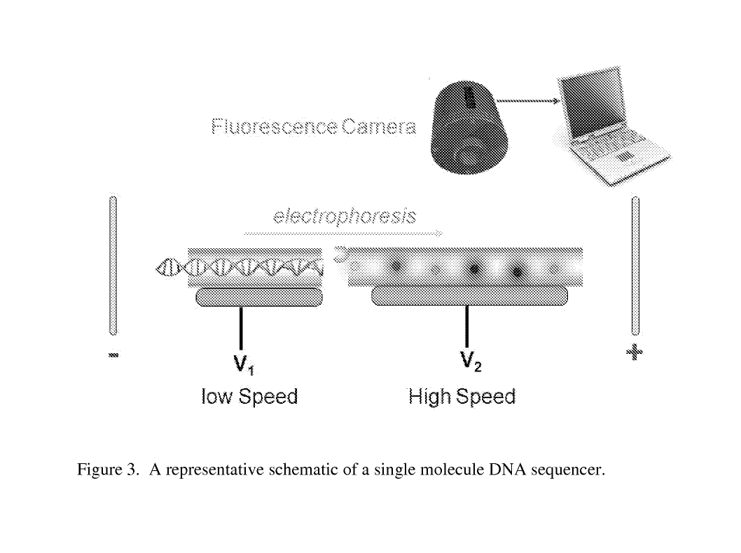

### DEFINITIONS

Unless specifically noted otherwise herein, the definitions of the terms used are standard definitions used in the art of microfluidics, nanofluidics, DNA sequencing and related technologies. Exemplary embodiments, aspects and variations are illustratived in the figures and drawings, and it is intended that the embodiments, aspects and variations, and the figures and drawings disclosed herein are to be considered illustrative and not limiting.

The fundamental difficulty of single molecule DNA sequencing lies on the fact that the distance between individual bases is beyond the resolution of currently available imaging techniques. DNA molecules have been observed in fast movement in applied electrical fields with mobility in solution 5×10⁻⁹ m²/(V·s). The spacing between two individual bases will be at the magnitude of 0.34 nm and less than 1 picosecond is needed for the macromolecule to translocate this distance with the above speed. Moreover, these macromolecules generally form complicated structures; for example, random coil as the secondary structure.

Most researchers have focused on improving the resolution to identify individual bases. In one embodiment, the present application provides a solution to this problem from a completely different perspective: to increase the spacing between individual monomers, oligomers, bases etc by controlling individual fragments of the macromolecule or the macromolecules, such as a polynucleotide molecule passing through nanochannels, particularly the translocation rate. In one aspect, the methods and designs disclosed in the present application will allow innovative techniques designed for the third generation single molecule based sequencing technology.

In one embodiment, the present application discloses methods and designs for controlling the movement of polymeric molecules or macromolecules, such as a single-stranded DNA (“ssDNA”), to expand the distance between the monomeric units that make up the polymer; for example, the individual nucleotides that compose the ssDNA, such that the individual nucleotides can be easily identified by conventional detection techniques.

In another embodiment, the present application discloses methods and designs of devices and apparatus that can increase throughput and decrease the time to obtaining results along with the concurrent decrease in cost. In another embodiment, the disclosed methods and designs may also be used to analyze properties or identity of other charged polymers and macromolecules, such as RNA and protein.

In one embodiment, the present method comprises of a method for passing or conveying a polymeric molecule or macromolecule, for example single-stranded DNA (“ssDNA”), through a nanoscopic opening, such as a nanochannel or nanotube, and controlling individual poly nucleotide molecule and/or fragment(s) of the molecule motion, by applying a gate field from internal electrodes or external electrodes. While maintaining the overall speed of the whole molecule, applying different gate fields to different zones of the nanochannel or tube can fine tune the relative speed among different parts or fragments (or segments) of the macromolecule. In one aspect, the method comprises the application of at least two different gate potential values to at least two different zones of the nanochannel in which the macromolecule resides, for example, resulting in a higher translocation speed of the fore (or front) segment of the macromolecule than that of the hind segment of the macromolecule in the nanochannel.

In another aspect, applying at least two different gate potentials in different zones of the nanochannel in which the macromolecule is present causes a change in the particular three dimensional structure of the macromolecule. By using this method, for example, because of the different intramolecular translocation speed of the different segments of the macromolecule, the secondary structure, such as a random coil of the macromolecule can be stretched out from the macromolecule's original structure or conformation to form a stretched out coil, a stretched semi-linear, a stretched linear configuration or combinations thereof. Together with other variables as described herein, for example the size of the nanochannel, the structure and motion different segments, and accordingly, the individual units, monomers or nucleotides of the macromolecules passing through the nanochannel can be finely controlled and maintained in a sequential, single file manner.

In another embodiment, upon passing through the different zones having different gate potentials, the macromolecule can then be cleaved into individual fragments and each fragment may be detected and identified. In one aspect, the macromolecule can be selectively cleaved into individual fragments. As desired, depending on the nature of the gate potentials applied to the system, the fragments may constitute an individual unit, monomer or nucleotide, or selected groups of dimers, trimers etc. For example, a DNA molecule may be hydrolyzed or cleaved by an exonuclease into individual nucleotides or bases that may be detected and identified using standard methods known in the art. The relative spacing among the fragments can be further increased and finely controlled by applying another gate potential to the yet another segment of the molecule. For example, another gate potential may be applied to the nanochannel after the exonuclease cleavage process.

In one aspect, the application of the proper gate potential values, when applied intramolecularly to the different segments of the macromolecule results in the different rates of translocation of the different segments of the macromolecule, and the spacing of the fragments can be significantly enlarged or extended, such that the spacing among individual fragments or nucleotides, for example, may be extended significantly into a range that can be resolved using currently available detection technologies. The individual fragments can then be detected, identified and analyzed, based on their chemical and physical properties. For example, the fragments may be detected using a charge-coupled device (CCD) to detect the optical signal from a single nucleotide with a fluorescent label attachment to identity the nucleotide base.

As a consequence of the process, the sequence of the macromolecule, such as a DNA molecule, may be obtained. The identity of the individual nucleotide base, for example, may also be detected as a modulation of the ionic current by the passage of a DNA molecule through a nanopore, or nanogap etc in the nano system, such as a microfluidic device. Overall, in the process of driving the polymer molecules to pass through the nanochannel, this nanoscopic system applies an electrical gate field, through external electrodes, and the translocation speed of different segments and fragments of the polymer may be controlled and fine-tuned. The mobility of the molecule may depend on various parameters, including the size of the molecule, electrical field, channel size, surface charge, surface coating, etc.

Previous experiments using nanopores have confirmed the translocation of polynucleotide molecules through nanometer size channels [1] and resulted in observable ionic current modulation. Further experiments also show that this translocation can occur even through 2 nm carbon nanotubes [2]. The macromolecule, for example ssDNA or fluorophore doped single strand DNA molecules, can be driven to pass through a nanotube or nanochannel, by an external driving potential by an electrophoresis process. In one aspect, the electrophoresis of fluorophore doped DNA molecules inside nanochannel may be observed in-situ with a fluorescence microscope.

One dimensional inorganic nanochannel may be used in the presently disclosed device or system. In order to slow down translocation of the negatively charged DNA molecule, the positively charged nanotube channel such as alumina tube may be employed rather than a negatively charged silica tube. These alumina nanotubes may be readily prepared with either nanowire template or self-sealing atomic layer deposition (ALD) process [3, 4]. As shown in the ionic field effect transistor, the ion motion inside the nanotube can be electrically modulated with external gates [3]. When positively gated, the positive charge on the nanochannel wall will strongly interact with negatively charged DNA backbone, thus slowing down its electrophoresis motion. The optimal condition for the polynucleotide molecule's slow and constant translocation may be finely controlled using the present process.

In another aspect, at least two separated external gate fields may be applied to at least two adjacent zones of the nanochannel, which may be patterned close to each other to locally alter the DNA mobility inside the channel. The patterning of the electrode may be performed using standard methods known in the art, including the method of electron beam lithography or for example, the method descried in U.S. Pat. Appl. Publ. 2007/0059645 A1.

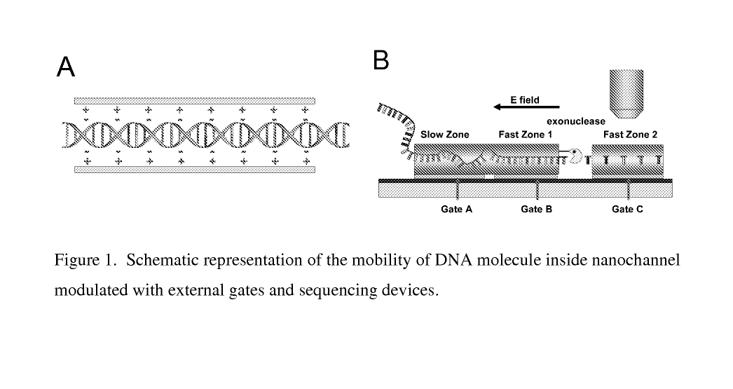

As shown in FIG. 1B, the worm-like single DNA molecule is stretched as it passes through the slow and fast zones. This stretching can be observed optically, for example, using fluorophore that may be doped or labeled onto DNA molecules inside nanochannel. The doped DNA molecules may be observed in-situ with a detector, such as a fluorescence microscope. In one aspect, the process may be used to study and examine the mechanical property of the single DNA molecule and to directly measure its length.

In another aspect, an exonuclease may be configured to the outlet of the nanochannel, such as the fore part of the nanochannel. This fore part may comprise multiple zones with different gate potentials. Application of the different gate potential results in the modification of the structure of the macromolecule. In one aspect, the passing of the macromolecule through the different zones having different gate potential results in the stretching or extension of the macromolecule. In one aspect, the zones may comprise at least two zones and the process provides the delivery of the molecule, such as a DNA molecule, in a stretched, linear or semi-linear form, and may be in sequential and/or single file order from the nanochannel.

The macromolecule, such as a DNA molecule, can be passed or conveyed through the nanochannel with finely controlled speed and the molecule may be cleaved or hydrolyzed into individual bases. In one aspect, these cleaved bases may be conveyed into the next zone, such as a fast zone, in the original order of the DNA sequence.

In one aspect, the device allows the significant increase the speed of single base motion in the second or subsequent fast zone that is up to about 1000-5000 times faster than the speed of the DNA molecule in slow zone, so that the distance between each base will be increased from 0.34 nm in DNA molecules to 300nm-1.5 μm or greater. With this increase in the spacing, the detector or optical system can readily resolve the individual fluorophore labeled base and thus directly determine DNA molecule's sequence.

Various parameters and conditions may be controlled to optimize the process. Non-limiting, representative conditions that may be controlled and optimized include:

The size of the nanotubes. For example, the internal diameter (ID) may be about 2-20 nm, and the thickness of the tube may be about 10-100 nm;

Application of external gate voltage range from 10-100 volt;

Modification of the driving potential, such as electrophoresis potential;

Nature, type and concentration of the solution used as the electrode solution, such as a 1 mol/L potassium chloride solution;

Applying the gate potential to control the translocation speed, which may be about 20 mm/sec, and may be decreased to 10-20 micrometer/sec in the slow zone;

Spacing or distance between bases: About 0.34 nm to 10 nm, 30 nm, 50 nm, 100 nm, 200 nm, 300 nm, 400 nm, 500 nm, 600 nm, 700 nm, 1 μm, 5 μm or greater;

For a single channel efficiency, the following representative variables or parameters may be employed:

Translocation speed may be >10 kbp/s;

Exonuclease cleavage number may be >1 kbp/s; and

CCD camera readout speed of about 100˜1 kbp/s; and permutations thereof.

In another aspect, the detection and characterizing the oligomers or individual nucleotides may be performed by passing the oligomer or nucleotide through a nanochannel decorated with functional groups on the surface. For example, a series of individual nucleotides, obtained in the original order or sequence in the DNA molecule, may pass through the nanochannel using the above cited methods.

In one aspect, the nanochannel may have the nucleotide attached to the surface. The ratio of the amount of attached nucleotide may be designed so that different nucleotides will gain different speed and acceleration upon passing through the nanochannel due to their different interactions with the decorating groups. Accordingly, by detecting the final speed of the individual nucleotide passing through the nanochannel, one can determine the identity of the nucleotide and thus, determine the sequence of the DNA molecule. Device Fabrication:

In one embodiment, as a representative example, the nanochannel based DNA sequencer may be fabricated as follows. Silicon nanowires, such as 10 nm diameter silicon wires, are grown on silicon <111> substrate by using 10 nm gold or platinum nanoparticles as catalyst in chemical vapor deposition furnace at 1000 K. Then the nanotubes may be prepared on this nanowire template with 40 nm alumina layer coating by atomic layer deposition followed by silicon etch in XeF₂ atmosphere. After dispersing the nanotube onto an insulating substrate, two 10 μm wide adjacent conductive gate electrodes are patterned on top of the nanotube lithographically with around 10 nm gap. The two different electrodes may define the slow and fast zone, respectively.

The nanotube may be cut at the gap by plasma etching where the two patterned electrodes serve as etching mask. Three micro reservoirs are patterned on the substrate which aligns to the inlet of slow zone, gap and outlet of fast zone, respectively, and connected to microfluidic system. The exonuclease is configured on the gap by introduce the enzyme solution into the second reservoir from the microfluidic channel or nanochannel.

To operate the DNA sequencer, the fluorescence labeled single strand DNA is introduced into the first reservoir and electrophoretically driven through the nanotube (or nanochannel) under 1-2 V bias. Around 10 V-20 V gate bias is applied to the slow zone electrode, which decreases the DNA translocation speed to ˜5-10 μm/s. After exit the slow zone, the DNA molecule will be cleaved into individual nucleotide bases and enters the second fast zone in the original order of the sequence. The fast zone electrode is bias at around 0 V, which enables the fast nucleotides translocation at ˜20 mm/s. Due to the difference in speed of the segments, the spacing between individual nucleotide bases can be greatly enhanced to around 1 μm. A single molecule fluorescent microscope is used to identify the DNA sequence by directly imaging the fast zone [5]. Furthermore, a label-free sequencing can also be realized by coupling this DNA sequencer, with nanochannels which is capable of distinguishing individual single nucleobases, for example nanopore [6].

This device is found to be very effective for the single molecule DNA sequencing. Combined with other parts, e.g. sample preparation, single molecule florescent microscope or electrical signal detection, the sequencer can achieve the following performance: Device efficiency for DNA sequencing; 10 k channel in parallel; Overall speed=10 k×100˜1 kbp/s=1M˜10 Mbp/s; and 1 hour˜8 hour per genome for 10× coverage.

While a number of exemplary embodiments, aspects and variations have been provided herein, those of skill in the art will recognize certain modifications, permutations, additions and combinations and certain sub-combinations of the embodiments, aspects and variations. It is intended that the following claims are interpreted to include all such modifications, permutations, additions and combinations and certain sub-combinations of the embodiments, aspects and variations are within their scope.

### REFERENCES

1. Salieb-Beugelaar, G. B., et al., Field-Dependent DNA Mobility in 20 nm High Nanoslits. Nano Letters, 2008. 8 (7): p. 1785-1790.

2. Liu, H., et al., Translocation of Single-Stranded DNA Through Single-Walled Carbon Nanotubes. Science, 2010. 327 (5961): p. 64-67.

3. Fan, R., et al., Polarity Switching and Transient Responses in Single Nanotube Nanofluidic Transistors. Physical Review Letters, 2005. 95 (8): p. 086607.

4. Nam, S.-W., et al., Sub-10-nm Nanochannels by Self-Sealing and Self-Limiting Atomic Layer Deposition. Nano Letters, 2010. 10 (9): p. 3324-3329.

5. Qu, X., et al., Nanometer-localized multiple single-molecule fluorescence microscopy. Proceedings of the National Academy of Sciences of the United States of America, 2004. 101 (31): p. 11298-11303.

6. Clarke, J., et al., Continuous base identification for single-molecule nanopore DNA sequencing. Nat Nano, 2009. 4 (4): p. 265-270.

The entire disclosures of all documents cited throughout this application are incorporated herein by reference.

What is claimed is:

1. A microfluidic device comprising:
at least one nanochannel comprising an inlet channel and an outlet channel, wherein the nanochannel comprises at least one slow zone and a first fast zone configured in the nanochannel;
the nanochannel further configured to receive a macromolecule at the inlet channel and the inlet channel configured to be in fluid communication with the slow zone, the first fast zone and the outlet channel;
wherein the macromolecule comprises a leading segment, a middle segment and an end segment;
at least one external electrode configured with the nanochannel for applying a first gate potential to the slow zone and a second gate potential to the first fast zone of the nanochannel for independently controlling and changing the rate of motion of at least among the leading segment, the middle segment and the end segment of the macromolecule, for controlling the rate of translocation of the macromolecule through the nanochannel and for controlling the rate of motion of a fragment obtained from a cleavage from the macromolecule;
the nanochannel further configured to provide contact of the macromolecule with an enzyme to cleave the macromolecule to form a fragment of the macromolecule; and
a detector for detecting and identifying a property of the fragment, wherein the detector is configured to detect the fragment in a second fast zone, wherein the fragment detected is obtained in the same sequence as the sequence of the uncleaved macromolecule.

2. The microfluidic device of claim 1, wherein the application of a first gate field to the slow zone upon the motion of the macromolecule through the nanochannel slows the motion of at least one segment of the macromolecule, and the application of a second gate field to the fast zone increases the motion or speed of at least one segment of the macromolecule, stretching the macromolecule segments between the slow zone and the first fast zone.

3. The microfluidic device of claim 1, wherein the fragment of the macromolecule is a monomer.

4. The microfluidic device of claim 1, wherein the macromolecule is deoxyribonucleic acid (DNA) or ribonucleic acid (RNA).

5. The microfluidic device of claim 1, wherein the first gate potential and the second gate potential are different.

6. The microfluidic device of claim 1, wherein the detector detects an optical signal capable of detecting a single molecule fluorescence and/or capable of detecting a single molecule Raman signal.

7. The microfluidic device of claim 1, wherein the detector detects the change or difference in the electrical properties.

8. The microfluidic device of claim 1, wherein the microfluidic device is configured to provide a controlled single molecule delivery.

9. The microfluidic device of claim 1, wherein the property of the fragment is selected from the groups consisting of mechanical strength, length or number of bases/residues in polymers or oligomers and/or in a mixture, transport properties, molecular electronic properties of the macromolecule.

10. A method for obtaining an identity of a target macromolecule comprising:
a) introducing the target macromolecule to a microfluidic device, wherein the microfluidic device comprises:
at least one nanochannel comprising an inlet channel and an outlet channel, wherein the nanochannel comprises at least one slow zone and a first fast zone configured in the nanochannel;
the nanochannel further configured to receive the target macromolecule at the inlet channel and the inlet channel configured to be in fluid communication with the slow zone, the first fast zone and the outlet channel;
wherein the target macromolecule comprises a leading segment, a middle segment and an end segment;
at least one external electrode configured with the nanochannel for applying a first gate potential to the slow zone and a second gate potential to the first fast zone of the nanochannel for independently controlling and changing the rate of motion of at least among the leading segment, the middle segment and the end segment of the target macromolecule, for controlling the rate of translocation of the target macromolecule through the nanochannel and for controlling the rate of motion of a fragment obtained from a cleavage from the target macromolecule;
the nanochannel further configured to provide contact of the target macromolecule with an exonuclease to cleave the target macromolecule to form a fragment of the target macromolecule; and
a detector for detecting and identifying a property of the fragment, wherein the detector is configured to detect the fragment in a second fast zone, wherein the fragment detected is obtained in the same sequence as the sequence of the uncleaved target macromolecule;
b) conveying the target macromolecule through the nanochannel by applying an electrical field or a differential pressure;
c) applying a first gate potential to the slow zone, and a second, different gate potential to the fast zone to control the relative motion of the leading segment, the middle segment and the end segment of the target macromolecule to stretch or increase the spacing among the segments;
d) contacting the stretched segment of the target macromolecule with an enzyme to cleave the target macromolecule to form a cleaved fragment;
e) conveying the cleaved fragment to a second fast zone; and
f) detecting the cleaved fragment and determining the sequence of the target macromolecule.

11. The method of claim 10, wherein the application of a first gate field to the slow zone upon the motion of the macromolecule through the nanochannel slows the motion of at least one segment of the macromolecule, and the application of a second gate field to the first fast zone increases the speed or motion of at least one segment of the macromolecule, stretching the macromolecule segments between the slow zone and the first fast zone.

12. The method of claim 11, wherein the increase in speed or motion is about 1000 to 5000 times faster than the speed of the macromolecule in the slow zone.

13. The method of claim 10, wherein the stretched segment of the macromolecule is increased from about 0.3 nm to about 300 nm-1.5 μm or greater.

14. The method of claim 10, wherein the target macromolecule has a translocation speed of about 20 mm/sec and decreases to about 10-20 um/sec in the slow zone.

15. (canceled)

16. The method of claim 10, wherein the macromolecule is DNA.

17. The method of claim 10, wherein the first gate potential and the second gate potential are different.

18. The method of claim 10, wherein the detector detect an optical signal capable of detecting a single molecule fluorescence and/or capable of detecting a single molecule Raman signal.

19. (canceled)

20. (canceled)

21. The method of claim 10, wherein the microfluidic device is configured to provide a controlled single molecule delivery.

22. The method of claim 10, wherein the property of the fragment is selected from the groups consisting of mechanical strength, length or number of bases or residues in polymers or oligomers and/or in a mixture, transport properties and molecular electronic properties of the macromolecule.

23. (canceled)

24. The method of claim 10, wherein the exonuclease cleaves the macromolecule at a rate of >1 kbp/s.
